(19) United States
(12) Patent Application Publication
(10) Pub. No.: US 2014/0324156 A1
(43) Pub. Date: Oct. 30, 2014

(54) IMPLANTABLE DEVICE WITH PLASMA POLYMER SURFACE

(71) Applicants: Yongbai Yin, Epping (AU); Marcela Bilek, Sutherland (AU); David McKenzie, Artarmon (AU)

(72) Inventors: Yongbai Yin, Epping (AU); Marcela Bilek, Sutherland (AU); David McKenzie, Artarmon (AU)

(73) Assignee: THE UNIVERSITY OF SYDNEY, Sydney (AU)

(21) Appl. No.: 14/125,120

(22) PCT Filed: Jun. 21, 2012
(86) PCT No.: PCT/AU2012/000714

§ 371 Date: Feb. 26, 2014

Related U.S. Application Data
(60) Provisional application No. 61/571,157, filed on Jun. 21, 2011.

(51) Int. Cl. A61L 31/10; A61L 31/16; A61L 31/14; A61F 2/82; A61L 31/02

(52) U.S. Cl. CPC A61L 31/10; A61F 2/82; A61L 31/022; A61L 31/14; A61L 31/16; A61F 2240/001; A61L 2420/04
USPC 623/1.15; 204/192.1

(57) ABSTRACT

A deformable implantable medical device such as a stent comprising metallic, polymer and/or composite substrate having a columnar structured plasma polymer surface capable of binding functional biological molecules. The polymer surface can be bound to the substrate through a mixed or graded interface formed by a co-deposition process where a substrate material is deposited with carbon containing species while gradually reducing the substrate material proportion and increasing the carbon containing species proportion. The device is able to undergo deformation without substantial delamination of the plasma polymer surface.

### FIELD OF THE INVENTION

The present invention relates in particular, but not exclusively, to deformable implantable medical devices comprising a columnar structured plasma polymer surface capable of binding functional biological molecules, wherein the devices are able to undergo deformation without substantial delamination of the plasma polymer surface. The invention also relates to methods of producing such devices. In particular, but not exclusively, the devices comprise stents.

### BACKGROUND OF THE INVENTION

It is desirable to be able to attach biological molecules strongly, preferably by means of a covalent bond, to surfaces of metals, polymers or composite materials that may be, or may be included as components within, implantable medical devices. For example, metals have desirable strength and elastic properties that make them suitable for use in repairing human and animal bones and joints. In particular, metal prosthetic pins and plates can be used to repair bone after fracture. In this context it is desirable to attach bone cells firmly to the metal surface so that the metal part is firmly anchored in the skeleton. For such applications it is desirable to promote the healthy growth of osteoblasts that give rise to bone and to suppress growth of fibroblasts that give rise to fibrous tissue. Such differentiation of cell attachment can be facilitated by attaching to the surface one or more suitable biologically active molecules. Another application of a metal prosthetic part is in stents for maintaining flow through blood vessels or other body cavities. Such devices should be biocompatible but should not promote excessive fibrous tissue or smooth muscle cell growth, whilst promoting the attachment and growth of endothelial cells. Such selectivity of cell type can also be attained by attaching suitable biological molecules to the metal surface.

It is also desirable to be able to covalently attach biological molecules to the surfaces of composite materials for purposes of skeletal repair, for the same reasons as outlined above in relation to metals. Indeed there are a variety of other applications for implants in which it is desirable to be able to covalently attach biological molecules to the surfaces of metals and polymers or to the surfaces of composite materials that have some metallic, ceramic and/or polymeric characteristics or features. For example, it is desirable to attach biological molecules to surfaces in the contexts of scaffolds for tissue and/or organ generation, sutures, staples, bone or tissue replacement materials, artificial organs, heart valves, replacement vessels and the like.

In earlier work (published in International patent publication. WO2009/015420, the disclosure of which is included herein in its entirety) from the same research group responsible for the present work a method was conceived that can be used to covalently bind functional biological molecules to a substrate, especially metal, semiconductor, polymer, ceramic or composite substrates, without the need to use linker molecules (and therefore without associated wet chemistry). This earlier invention involves a simple two step plasma modification process including ion implantation and/or deposition, to create a mixed or graded interface, followed by the deposition of a hydrophilic plasma polymer. The binding of biological molecules then involves simple adsorption (resulting in covalent binding), with no further chemistry required.

The inventors have, however, identified a problem with the generation of biologically functionalised surfaces in the context of deformable implants. Deformable implants, such as stents, require significant mechanical strength. Unfortunately though, the types of materials that possess acceptable mechanical strength, such as metals and composite materials, are generally not biologically compatible. For example, specifically in the case of metallic stents, the metallic surface thus needs to be modified along the lines outlined above so that a biomimetic protein layer can be strongly attached and a biocompatible surface can be achieved. Although a biocompatible layer can be generated using deposition of a plasma polymer onto substrates such as metal stents to provide a biocompatible surface, the inventors have found that there can be problems associated with insufficient adhesion of the surface coating leading to delamination, particularly in regions where the coating is subject to high levels of mechanical strain due to deformation of the substrate. The surfaces coated on deformable implants should not only demonstrate a good capacity for binding to biological molecules such as proteins, but also have sufficient mechanical reliability and adhesion of the coating to the substrate to substantially overcome the problem of delamination of the coating. Such mechanical failures are well-known and are particularly problematic as delamination not only compromises biocompatibility of the implant, but can result in a release of fragments of the coating material. In the case of stents such problems are acute as they can lead to the formation of blood clots and/or blockages of the vasculature. Adhesion problems between substrate layers and plasma polymers are essentially due to the contrasting properties of the two materials and very high mechanical strains introduced by deformation of the implant such as would occur in the crimping and expansion of a vascular stent during deployment in the vasculature using a balloon angioplasty procedure.

The present inventors have now determined that producing a columnar structure of the substrate coating allows the coating to accommodate the strain to which an implant is subjected resulting from mechanical deformation, without substantial delamination of the coating. Other aspects and advantages of the present invention will become apparent from the following detailed description.

### SUMMARY OF THE INVENTION

According to one embodiment of the present invention there is provided a deformable implant comprising a metallic substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is a stent that is able to undergo deformation without substantial delamination of the plasma polymer surface.

According to another embodiment of the invention there is provided a deformable implant comprising a metallic, polymer and/or composite substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is able to undergo deformation without substantial delamination of the plasma polymer surface.

According to a further embodiment of the present invention there is provided a deformable implantable medical device comprising a columnar structured plasma polymer surface capable of binding functional biological molecules, wherein the device is able to undergo deformation without substantial delamination of the plasma polymer surface.

In one embodiment, the medical device is a stent.

For example, the columnar structures within the plasma polymer surface can have an average diameter of from about 10 nm to about 500 nm, from about 20 nm to about 300 nm or from about 30 nm to about 200 nm.

In another embodiment the mixed or graded interface of the deformable implant or medical device is also columnar structured.

According to a further embodiment of the present invention there is provided a method of producing a deformable implant comprising a metallic, polymer and/or composite substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is able to undergo deformation without substantial delamination of the plasma polymer surface, the method comprising exposing a surface of the substrate to co-deposition under conditions in which substrate material is deposited with carbon containing species while gradually reducing substrate material proportion and increasing carbon containing species proportion, and wherein the co-deposition is conducted under conditions that substantially eliminate energetic ion bombardment.

In one aspect the method can further involve incubating the activated columnar structured and hydrophilic plasma polymer surface with a functional biological molecule.

### BRIEF DESCRIPTION OF THE FIGURES

The invention will be further described with reference to the figures, wherein:

FIG. 1 shows the simulated stress distribution after a stent has been exposed to a crimping procedure. The white coloured stent component shows the stress distribution before crimping, and the coloured one stent component shows the stress distribution after crimping. The colour bar represents the scale of stress increasing from left to right.

FIG. 2 shows a schematic diagram of the reactive sputtering system used to deposit a mixed or graded interface on a substrate surface.

FIG. 3 shows a SEM image demonstrating the adhesion failure of a biocompatible surface deposited onto the stent without introducing a graded interface. The failure was analysed after receiving crimping and expansion. The adhesion failure is of the typical catastrophic type commonly seen in practice.

FIG. 4 shows a SEM image of a biocompatible surface deposited with graded interface on a component of a stent, after receiving crimping and expansion. Some adhesion failure is in evidence at a fraction of the area with graded interface, which suggests improvement of adhesion is necessary.

FIG. 5 shows a SEM image of biocompatible surface deposited on a stent with columnar structured graded interface, after receiving crimping and expansion. There is excellent adhesion, without evidence of delamination of the deposited surface coating.

FIG. 6 shows a high magnification SEM image of biocompatible surface deposited on a stent with columnar structured graded interface, after receiving crimping and expansion. In this image the stress accommodation mechanism can be visualised at the boundaries of the columnar structures.

FIG. 7 shows a high magnification SEM image of a biocompatible surface deposited on a stent with columnar structured graded interface. In this image the stress accommodation mechanism can be visualised at the boundaries of the columnar structures.

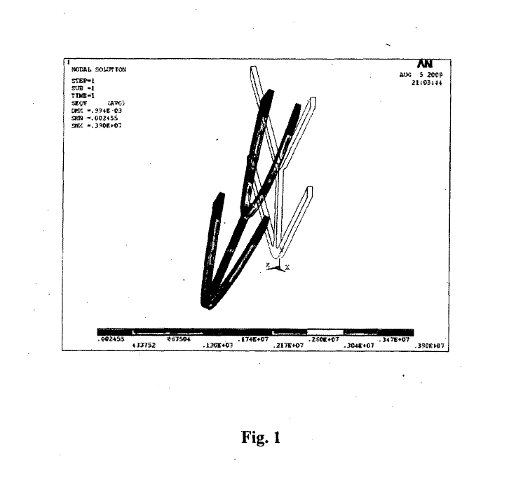

FIG. 1 has been filed in colour and is available on request.

### DETAILED DESCRIPTION OF THE INVENTION

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Documents referred to within this specification are included herein in their entirety by way of reference.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

As mentioned above, in one broad embodiment this invention relates to deformable implant comprising a metallic, polymer and/or composite substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is able to undergo deformation without substantial delamination of the plasma polymer surface. The deformable implants can themselves comprise an implantable medical device or they can comprise a component of an implantable medical device. For example, deformable medical devices contemplated by the invention include stents (such as vascular and gastrointestinal stents), prostheses, artificial joints, bone or tissue replacement materials or scaffolds, artificial organs, heart valves, replacement vessels, sutures and staples and other tissue or bone fixing or closing devices that undergo some form of deformation. In this context the term “deformation” is intended to imply that the implant or medical device undergoes some change in its physical configuration or conformation in the course of its preparation for implantation, implantation or use that gives rise to mechanical strain on elements of its surface. A prime example is the example of the crimping and expansion of a stent, but there is potentially similar deformation in the moving elements of a replacement joint, in the crimping of a staple or the bending and knotting of a suture, which are mentioned by way of example only.

Throughout this specification the deformable implants or deformable medical devices are referred to, when wishing to distinguish them from the coating that is applied to them, as the “substrate” for application of a surface coating.

By the term “activated” it is intended to mean that the hydrophilic surface layer (which also results from the plasma polymer deposition approach as disclosed in International patent publication WO2009/015420) on the metal, polymer or composite substrate has been processed or generated in a manner such that it is able to accept a biological molecule for binding, upon exposure thereto. That is, the surface layer on substrate has one or more higher energy state regions where there are chemical groups or electrons available for participation in binding to one or more groups on a biological molecule, or indeed to suitable linker groups, which in turn are bound or are able to bind to a biological molecule.

In another broad aspect of the invention there is provided a functionalised deformable implant or medical device wherein the substrate is bound through a mixed or graded interface to a hydrophilic plasma polymer surface that is directly covalently bound to a functional biological molecule, and wherein the plasma polymer surface comprises a sub-surface that includes a plurality of cross-linked regions.

Without wishing to be bound by theory, the present inventors believe that through the activation of the plasma polymer surface layer on the substrate it is possible to form chemical bonds, most likely covalent bonds, to chemical groups of biological molecules or linkers that attach to biological molecules. Preferably the chemical groups of the biological molecules are accessible for binding interactions, such as by being located on the exterior of the molecule. The present inventors believe that activation of the plasma polymer surface involves the generation of reactive free radicals or oxygen species, such as charged oxygen atoms and reactive carbonyl and carboxylic acid moieties that appear following exposure of the plasma treated or generated polymer surface to oxygen (e.g. from air), and which are then available as binding sites for reactive species on biological molecules, such as amine groups. The most likely mechanism to explain activation of the plasma polymer surface layer on the substrate is that the methods of the invention give rise to the generation of free radicals within the plasma polymer surface. Indicative of this mechanism is that while the biological activity of biological molecules with which the surface has been functionalised is retained over time, there appears to be a loss over time of the ability of activated surfaces to bind covalently to biological molecules. However, the ability to bind biological molecules to the activated surfaces can be regenerated (that is, the previously activated surfaces can effectively be re-activated) by adopting an annealing step. This is a step of applying energy to the surface, without destroying it, to allow molecular mobility within the plasma polymer such that buried free radicals can migrate to the surface where they can participate in covalent binding to biological molecules. Alternatively, the energy applied may release bound chemical species that, once released, give rise to free radicals. For example, annealing may be carried out by heating in an oven or exposure to steam or microwave energy (for example to temperatures of 250° C. to 400° C., 300° C. to 375° C., or approximately 350° C., depending upon the surface concerned). A preferred method of annealing is heating in a vacuum oven. The annealing step may be undertaken as part of the manufacture of the activated surface. For example this step may ensure that the activation is at a high level even if the manufacturing process is not fully optimised.

Within this specification we refer to attachment of a biological molecule, or a linker for attachment to a biological molecule, as functionalisation of the plasma polymer surface on the substrate and to the plasma polymer surface on the substrate to which the biological molecule or linker is attached as being “functionalised”. Attachment by covalent bonds to an otherwise hydrophilic surface allows strong time stable attachment of biological molecules that are able to maintain a useful biological function. For example, the hydrophilic surface of the plasma polymer layer will ensure that it is not energetically favourable for proteins to denature on the surface. Covalent attachment of a protein to a surface can be achieved via amino acid side chain groups of the protein covalently attached to the surface or to linker molecules, for example. The strategy adopted is to prepare the plasma polymer surface with sites that encourage covalent attachment. Using functionality assays, the inventors have demonstrated that associated with the adopted plasma surface treatment there is enhancement of functional protein attachment with covalent binding, compared to non-treated surfaces, as well as significantly increased resistance to repeated washing steps. That is, there is increased biological molecule binding relative to non-treated surfaces, the binding is strong and can withstand repeated washing and the molecule is able to retain useful activity (i.e. the biological molecule is functional or retains some useful functionality).

By the term “functional” it is intended to convey that the molecule is able to exhibit at least some of the activity it would normally exhibit in a biological system. For example, activity may include the maintained ability to participate in binding interactions, such as antigen/antibody binding, receptor/drug binding, the maintained ability to catalyse or participate in a biological reaction or the ability to interact with cell membrane proteins in biological tissues even if this is at a lower level than is usual in a biological system. Routine assays are available to assess functionality of the biological molecule. Preferably the activity of the biological molecule bound to the activated plasma polymer surface is at least 20%, preferably at least 40%, more preferably at least 60%, 70% or 80% and most preferably at least 90%, 95%, 98% or 99% of the activity of the molecule when not bound to the activated plasma polymer surface. Most preferably the activity of the bound biological molecule is equivalent to that of a non-bound molecule.

By the term “biological molecule” it is intended to encompass any molecule that is derived from a biological source, is a synthetically produced replicate of a molecule that exists in a biological system, is a molecule that mimics the activity of a molecule that exists in a biological system or otherwise exhibits biological activity, or active fragments thereof. The term “biological molecule” also encompasses a combination or mixture of biological molecules. In the case of proteins (and a similar analogy can be made in the case of nucleic acids, carbohydrates or the like) active fragments are peptide sequences derived from the active protein that exhibit preferably at least at least 20%, preferably at least 40%, more preferably at least 60%, 70% or 80% and most preferably at least 90%, 95%, 98% or 99% of the activity of the active protein. Active peptide fragments are preferably at least 4, more preferably at least 10, more preferably at least 15, 20, 30, 40 or 50 amino acids in length. Examples of biological molecules include, but are not limited to, amino acids, peptides, enzymes, proteins, glycoproteins, lipoproteins, nucleotides, oligonucleotides, nucleic acids (including DNA and RNA), lipids and carbohydrates, as well as active fragments thereof. Preferred biological molecules include proteins and drugs or drug targets. Particularly preferred biological molecules include antibodies and immunoglobulins, receptors, extra-cellular matrix proteins, enzymes, neurotransmitters or other cell signalling agents, cytokines, hormones and complementarity determining proteins, and active fragments thereof. The term “biological molecule” also encompasses molecules that are integral to or attached to cells or cellular components (e.g. cell membrane proteins) through which cells or cellular components may be bound to the activated plasma polymer. A biological molecule of particular interest is tropoelastin, which is an extracellular matrix protein that can be used to functionalise surfaces to improve the biological compatibility of implantable or other devices. Enzymes of interest include those capable of breaking down cellulose into simple sugars such as cellulase.

An advantage associated with the present invention is that the process for binding biological molecules to the surface of a substrate does not depend upon the specific biological molecule or the nature of the substrate and can therefore be applied to a wide variety of biological molecules and substrates. Furthermore, and although it is possible for the biological molecules to be bound via a linker molecule, it is not necessary according to the present invention for linker molecules to be utilised, which means that time consuming and potentially costly and complex wet chemistry approaches for linkage are not required.

An important element of the present invention is that the present inventors have determined that by providing a columnar structure in the surface coating of a deformable implant or medical device it is possible to address the problem of delamination of the surface coating that may otherwise occur in regions subject to mechanical strain resulting from deformation. The introduction of a columnar structured surface gives rise to nanoscale gaps or crevices between adjacent columns so that mechanical stress within the surface coating can be accommodated by expansion or contraction of the gaps or crevices and delamination resulting from deformation is eliminated or at least substantially reduced relative to the effect that may be observed by microscopy (for example Scanning Electron Microscopy (SEM)) in an equivalent implant or device with a surface coating that is not columnar structured. That is, the invention allows deformation of the implant or device without substantial delamination or peeling off of the surface coating.

In some embodiments of the invention the substrate has a mixed or graded interface onto which a plasma polymer surface is deposited. While the inventors have observed that good results in terms of eliminating deformation induced delamination can be obtained when both the mixed or graded interface and the plasma polymer surface are columnar structured, there is some benefit in terms of minimising delamination when a columnar structured plasma polymer surface is deposited on a non-columnar mixed or graded interface. This possibility is therefore also encompassed by the invention.

In one embodiment, the mixed or graded interface is columnar structured. In other words, the mixed or graded interface may be described as a columnar structured mixed or graded interface.

It is also possible for a non-columnar plasma polymer surface to be deposited onto a columnar mixed or graded interface. However, without wishing to be bound by theory, the inventors expect that in this case deformation will result in fracturing of the plasma polymer surface so that it takes on aspects of the appearance or characteristics of a plasma polymer surface that is deposited in a columnar structured form. Where the context permits, reference to the columnar structured plasma polymer surface throughout this specification is intended to encompass this possibility.

By the term “columnar structure” it is intended to convey that the surface concerned has the appearance at a microscopic scale of an array of projections or columns emanating form the surface. Each projection can have a substantially consistent diameter throughout its cross section or the projections can taper towards their distal ends. Preferably, however, the projections are relatively tightly packed so that there is a columnar structured array on coated regions of the substrate that largely prevents biological materials from accessing the substrate surface. The cross sectional shape of the projections is likely to be random and although it is not essential for the projections to be of consistent size the best results in terms of minimising deformation induced delamination are expected to be achieved when the cross sectional diameter of the projections or columns is of a similar order across the columnar structured array. In one aspect of the invention the columnar structures or projections within the columnar structured surface will have an average cross sectional diameter (assuming that circles of best fit are placed over the projections in order to make this calculation and to take account of the random shapes of the projections) of from about 10 nm to about 500 nm, for example from about 20 nm to about 300 nm or from about 30 nm to about 200 nm, about 30 nm to about 150 nm, about 35 nm to about 100 nm or about 40 nm to about 80 nm.

As indicated above the present invention can be utilised to attach functional biological molecules to surfaces of a wide variety of deformable implant or medical devices, which will also be referred to herein simply as “substrates”. For example the substrate may take the form of a block, sheet, film, foil, tube, strand, fibre, shaped article, indented, textured or moulded article or woven fabric or massed fibre pressed into a sheet (for example like paper) of metal, polymer or composite. The substrate can be a solid mono-material, laminated product, hybrid material or alternatively a coating on any type of base material which can be non-metallic or metallic in nature, and which may include a polymer component, such as homo-polymer, co-polymer or polymer mixture.

Throughout this specification the term “plasma polymer” is intended to encompass a material produced on a surface by deposition from a plasma, into which carbon or carbon containing molecular species are released. The carbon containing molecular species are fragmented in the plasma and a plasma polymer coating is formed on surfaces exposed to the plasma. This coating contains carbon in a non-crystalline form together with other elements from the carbon containing molecular species or other species co-released into the plasma. The surface may be heated or biased electrically during deposition. Such materials often contain unsatisfied bonds due to their amorphous nature.

The term “hydrophilic” refers to a surface that can be wetted by polar liquids such as water, and include surfaces having both strongly and mildly hydrophilic wetting properties. For a smooth surface we use the term hydrophilic to mean a surface with water contact angles in the range from 0 to around 90 degrees. The most preferable water contact angle for the hydrophilic surfaces relating to the present invention are in the range of around 50 to about 70 degrees.

As a result of the plasma treatment according to the invention the present inventors have determined that not only is the substrate surface activated to allow binding of one or more biological molecules, but that the possibly hydrophobic nature of the surface is modified to exhibit a more hydrophilic character. This is important for maintaining the conformation and therefore functionality of many biological molecules, the outer regions of which are often hydrophilic in nature due to the generally aqueous environment of biological systems. The inventors have also shown that not only do techniques of the present invention give rise to hydrophilicity of the treated substrate, but that as a result of cross linked sub-surface regions in the plasma polymer there is a delay to the hydrophobic recovery of the surface that takes place over time following the treatment. The term “sub-surface” is intended to encompass a region of the plasma polymer, which may be the entire interior of the plasma polymer layer or part thereof subject to plasma deposition, that is between about 0.5 nm and about 1000 nm beneath the final coating surface, preferably between about 5 nm and about 500 nm, 300 nm or 200 nm, and most preferably between about 10 nm and about 100 nm beneath the surface.

The term “polymer” as it is used herein is intended to encompass homo-polymers, copolymers, polymer containing materials, polymer mixtures or blends, such as with other polymers. The term “polymer” encompasses thermoset and/or thermoplastic materials, as well as polymers generated by plasma deposition processes. The term “polymer” also encompasses polymer like surfaces that include reactive species or electrons and which may approach, generally or in isolated regions, the appearance and structure of amorphous carbon. The columnar structured polymer surfaces may fully or partially coat or cover the substrate, may include gaps or apertures and/or regions of varied thickness.

The plasma polymer surface created in the process can be generated through plasma ion implantation with carbon containing species or co-deposition under conditions in which substrate material is deposited with carbon containing species while gradually reducing substrate material proportion and increasing carbon containing species proportion. In this context the carbon containing species may comprise charged carbon atoms or one or more other simple carbon containing molecules such as carbon dioxide, carbon monoxide, carbon tetrafluoride or optionally substituted branched or straight chain C₁ to C₁₂ alkane, alkene, alkyne or aryl compounds as well as one or more compounds more conventionally thought of in polymer chemistry as monomer units for the generation of polymer compounds, such as n-hexane, allylamine, acetylene, ethylene, methane and ethanol. Additional suitable compounds may be drawn from the following non-exhaustive list: butane, propane, pentane, heptane, octane, cyclohexane, cycleoctane, dicyclopentadiene, cyclobutane, tetramethylaniline, methylcyclohexane and ethylcyclohexane, tricyclodecane, propene, allene, pentene, benzene, hexene, octene, cyclohexene, cycloheptene, butadiene, isobutylene, di-para-xylylene, propylene, methylcyclohexane, toluene, p-xylene, m-xylene, o-xylene, styrene, phenol, chlorphenol, chlorbenzene, fluorbenzene, bromphenol, ethylene glycol, diethylene glycol, dimethyl ether, 2,4,6-trimethyl m-phenylenediamine, furan, thiophene, aniline, pyridine, benzylamine, pyrrole, propionitrole, acrylonitrile, pyrrolidine, butylamine, morpholine, tetrahydrofurane, dimethylformamide, dimethylsulfoxide, glycidyl methacrylate, acrylic acid, ethylene oxide, propylene oxide, ethanol, propanol, methanol, hexanol, acetone, formic acid, acetic acid, tetrafluormethane, fluorethylene, chloroform; tetrachlormethane, trichlormethane, trifluormethane, vinyliden chloride, vinyliden fluoride, hexamethyldisiloxane, triethylsiloxane, dioxane, perfluoro-octane, fluorocyclobutane, octafluorocyclobutane, vinyltriethoxysilane, octafluorotoluene, tetrafluoromethane, hexamethyldisiloxane, heptadecafluoro-1-decene, tetramethyldisilazane, decamethyl-cyclopentasiloxane, perfluoro(methylcyclohexane), 2-chloro-p-xylene.

In one aspect the plasma polymer surface has a thickness of from about 0.3 nm to about 1000 nm, from about 3 nm to about 500 nm, 300 nm or 100 nm or from about 10 nm to about 30 nm.

The terms “metal” or “metallic” as used herein to refer to elements, alloys or mixtures which exhibit or which exhibit at least in part metallic bonding. Preferred metals according to the invention include elemental iron, copper, zinc, lead, aluminium, titanium, gold, platinum, silver, cobalt, chromium, vanadium, tantalum, nickel, magnesium, manganese, molybdenum tungsten and alloys and mixtures thereof. Particularly preferred metal alloys according to the invention include cobalt chrome, nickel titanium, titanium vanadium aluminium and stainless steel.

The term “ceramic” as it is used herein is intended to encompass materials having a crystalline or at least partially crystalline structure formed essentially from inorganic and non-metallic compounds. They are generally formed from a molten mass that solidifies on cooling or are formed and either simultaneously or subsequently matured (sintered) by heating. Clay, glass, cement and porcelain products all fall within the category of ceramics and classes of ceramics include, for example, oxides, silicates, silicides, nitrides, carbides and phosphates. Particularly preferred ceramic compounds that may be included within composite materials encompassed in the invention include magnesium oxide, aluminium oxide, hydroxyapatite, titanium nitride, titanium carbide, aluminium nitride, silicon oxide, zinc oxide and indium tin oxide.

“Composite” materials comprehended by the present invention include those that are combinations or mixtures of other materials, such as composite metallic/ceramic materials (referred to as “cermets”) and composites of polymeric material including some metallic or ceramic content, components or elements. Such composites may comprise intimate mixtures of materials of different type or may comprise ordered, arrays or layers or defined elements of different materials.

The term “polymer” as it is used herein is intended to encompass homo-polymers, copolymers, polymer containing materials, polymer mixtures or blends, such as with other polymers and/or natural and synthetic rubbers, as well as polymer matrix composites, on their own, or alternatively as an integral and surface located component of a multi-layer laminated sandwich comprising other materials e.g. polymers, metals or ceramics (including glass), or a coating (including a partial coating) on any type of substrate material. The term “polymer” encompasses thermoset and/or thermoplastic materials as well as polymers generated by plasma deposition processes.

The polymeric substrates which can be treated according to the present invention include, but are not limited to, polyolefins such as low density polyethylene (LDPE), polypropylene (PP), high density polyethylene (HDPE), ultra high molecular weight polyethylene (UHMWPE), blends of polyolefins with other polymers or rubbers; polyethers, such as polyoxymethylene (Acetal); polyamides, such as poly(hexamethylene adipamide) (Nylon 66); polyimides; polycarbonates; halogenated polymers, such as polyvinylidenefluoride (PVDF), polytetra-fluoroethylene (PTFE) (Teflon™), fluorinated ethylene-propylene copolymer (FEP), and polyvinyl chloride (PVC); aromatic polymers, such as polystyrene (PS); ketone polymers such as polyaryletherketone (PAEK) and polyetheretherketone (PEEK); methacrylate polymers, such as polymethylmethacrylate (PMMA); polyesters, such as polyethylene terephthalate (PET); and copolymers, such as ABS and ethylene propylene diene mixture (EPDM). Preferred polymers include polyethylene, PEEK and polystyrene.

The term “co-deposition” as used herein refers to a deposition process which deposits at least two species on a surface simultaneously, which may involve varying over time the proportions of the two or more components to achieve graded layers of surface deposition. Most preferably the deposition of this graded layer is commenced with deposition of only the substrate material, noting that layers deposited prior to the deposition of carbon containing species become the effective substrate.

By the term “mixed or graded interface” it is intended to denote a region in the material in which the relative proportions of two or more constituent components vary gradually according to a given profile. One method by which this mixed or graded interface is generated is by co-deposition, where the power supplied to the magnetron or cathodic arc source of metal, or the composition of the gases supplied to the process chamber are varied so that the deposited and/or implanted material changes progressively, for example, from more metallic to more polymeric.

The term “plasma” or “gas plasma” is used generally to describe the state of ionised vapour. A plasma consists of charged ions, molecules or molecular fragments (positive or negative), negatively charged electrons, and neutral species. As known in the art, a plasma may be generated by combustion, flames, physical shock, or preferably, by electrical discharge, such as a corona or glow discharge. In radiofrequency (RF) discharge, a substrate to be treated is placed in a vacuum chamber and vapour at low pressure is bled into the system. An electromagnetic field generated by a capacitive or inductive RF electrode is used to ionise the vapour. Free electrons in the vapour absorb energy from the electromagnetic field and ionise vapour molecules, in turn producing more electrons.

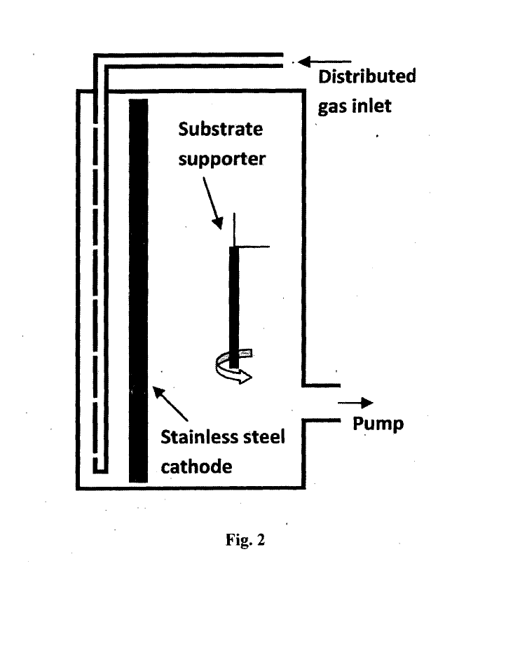

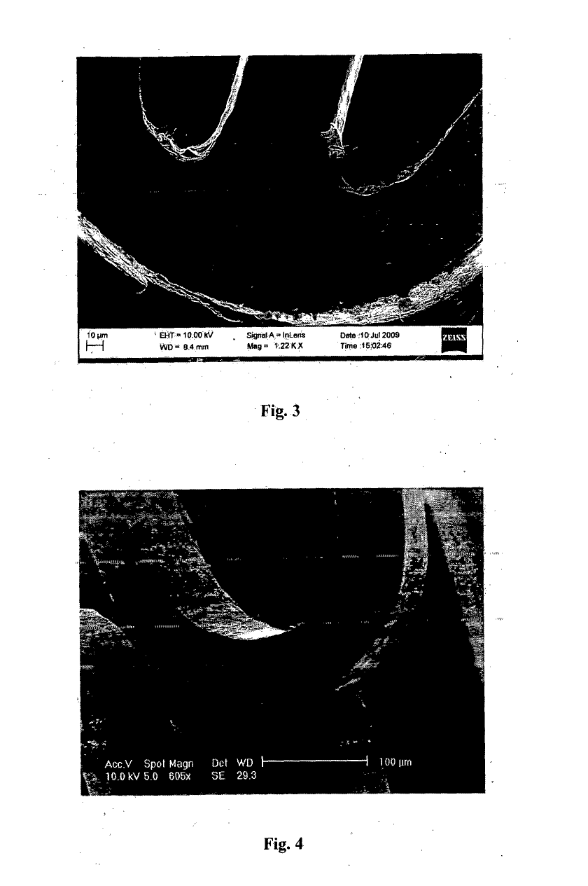

In conducting the plasma treatment according to the invention, typically a plasma treatment apparatus (such as one incorporating a Helicon, parallel plate or hollow cathode plasma source or other inductively or capacitively coupled plasma source, such as shown in FIG. 2) is evacuated by attaching a vacuum nozzle to a vacuum pump. A suitable plasma forming vapour, generated from a vapour, liquid or solid source is bled into the evacuated apparatus through a gas inlet until the desired vapour pressure in the chamber and differential across the chamber is obtained. An RF electromagnetic field is generated within the apparatus by applying current of the desired frequency to the electrodes from an RF generator. Ionisation of the vapour in the apparatus is induced by the electromagnetic field, and the resulting plasma modifies the metal, polymer or composite substrate surface subjected to the treatment process.

In one embodiment of the invention it is possible to treat the plasma polymer surface either while it is being deposited or after its deposition, with a plasma forming gas or vapour to thereby activate the plasma polymer surface for binding to biological molecules. Suitable plasma forming vapours used to treat the plasma polymer surface of the substrate include inorganic and/or organic gases/vapours. Inorganic gases are exemplified by helium, argon, nitrogen, neon, water vapour, nitrous oxide, nitrogen dioxide, oxygen, air, ammonia, carbon monoxide, carbon dioxide, hydrogen, chlorine, hydrogen chloride, bromine cyanide, sulfur dioxide, hydrogen sulfide, xenon, krypton, and the like. Organic gases are exemplified by methane, ethylene, n-hexane, benzene, formic acid, acetylene, pyridine, gases of organosilane, allylamine compounds and organopolysiloxane compounds, fluorocarbon and chlorofluorocarbon compounds and the like. In addition, the gas may be a vaporised organic material, such as an ethylenic monomer to be plasma polymerised or deposited on the surface. These gases may be used either singly or as a mixture of two more, according to need. Preferred plasma forming gases according to the present invention are argon and organic precursor vapours as well as inorganic vapours consisting of the same or similar species as found in the substrate.

Typical plasma treatment conditions (which are quoted here with reference to the power that may be required to treat a surface of 100 square centimetres, but which can be scaled according to the size of the system) may include power levels from about 1 watt to about 1000 watts, preferably between about 5 watts to about 500 watts, most preferably between about 30 watts to about 300 watts (an example of a suitable power is forward power of 100 watts and reverse power of 12 watts); frequency from 0 kHz (that is, dc) to about 10 GHz, preferably dc or about 15 kHz to about 50 MHz, more preferably from about 1 MHz to about 20 MHz (an example of a suitable frequency is about 13.5 MHz); axial plasma confining magnetic field strength of between about 0 G (that is, it is not essential for an axial magnetic field to be applied) to about 1000 G, preferably between about 20 G to about 500 G, most preferably between about 40 G to about 60 G (an example of a suitable axial magnetic field strength is about 50 G); exposure times of about 5 seconds to 12 hours, preferably about 1 minute to 2 hours, more preferably between about 5 minutes and about 20 minutes (an example of a suitable exposure time is about 13 minutes); gas/vapour pressures of about 0.0001 to about 10 torr, preferably between about 0.0005 torr to about 0.5 torr, most preferably between about 0.01 torr and about 0.2 torr (an example of a suitable pressure is about 0.15 torr); and a gas flow rate of about 1 to about 2000 cm³/min.

Following activation of the substrate surface it is possible to functionalise the plasma polymer surface with a biological molecule or linker by simple incubation (e.g. by bathing, washing, stamping, printing or spraying the surface) of the activated surface (substrate) with a solution comprising the biological molecule or linker. Preferably the solution is an aqueous solution (e.g. saline), that preferably includes a buffer system compatible with maintaining the biological function of the molecule, such as for example a phosphate or Tris buffer. It may then be appropriate to conduct one or more washing steps also using a biologically compatible solution or liquid, for example the same aqueous buffered solution as for the incubation (but which does not include the biological molecule), to remove any non-specifically bound material from the surface, before the functionalised plasma polymer substrate is ready to be put to its intended use. In another embodiment it is possible to use an agent such as bovine serum albumin (BSA) that will inhibit non-specific adsorption of further biological molecules.

The inventors have determined that both the activated substrates and the substrates functionalised with biological molecules according to the invention exhibit extensive shelf life. For example, the activated polymer coated substrate may be stored (preferably in a sealed environment) for a period of minutes, hours, days, weeks months or years before incubation with a biological molecule to result in functionalisation of the plasma polymer surface. De-activation takes place over time so that a longer incubation time is required to achieve a given level of protein attachment. This can be reversed by annealing, as discussed above. Similarly the substrates functionalised with biological molecules according to the invention may be stored (preferably following freeze drying and more preferably in a sealed environment at low temperature) for periods of minutes, hours, days, weeks, months or years without significant degradation before being re-hydrated, if necessary, and put to their intended use. If freeze drying is adopted, a stabiliser such as sucrose may beneficially be added before the freeze drying process. The sealed environment is preferably in the presence of a desiccant and may comprise a container or vessel (preferably under vacuum or reduced oxygen atmosphere) or may for example comprise a polymer, foil and/or laminate package that is preferably vacuum packed. Preferably the sealed environment is sterile to thus prevent or at least minimise the presence of agents such as proteases and nucleases that may be detrimental to activity of the biological molecules. Alternatively the activated or functionalised substrates may be stored in a conventional buffer solution, such as mentioned above.

The invention will now be described further, and by way of example only, with reference to the following non-limiting examples.

### EXAMPLES

### Example 1

### Deposition of Plasma Polymer Coating with Columnar Structure on Stent Surfaces with Mixed or Graded Interfaces

### Methods

All plasma polymerization experiments were conducted without heating or cooling of the surface. In the graded interface formation, we used a reactive sputtering method, in which the cathode material was 316L stainless steel. A schematic diagram of the system is shown in FIG. 2. The long cylindrical cathode (2 m) provided a wide variation in the angle of incidence of the depositing material in the vertical plane. The substrate holder was rotated to give a 180 range in the angle of incidence in the horizontal plane. This wide range of angles is important to ensure conformal coverage of complex-shaped objects. Argon and acetylene were used in the reactive sputtering and were injected into the chamber through a distributed gas line with an array of holes at 10 cm intervals. To form the graded interface, a pure stainless steel coating was first deposited onto the stainless steel substrates, followed by a coating that contained gradually increasing fractions of plasma polymer. To achieve this, the acetylene flow rate was increased from zero until a pure plasma polymer layer was formed. The initial sputtering voltage during the deposition of pure metal was 800V while the cathode current was maintained at 3 A. Increasing the flow rate of acetylene while keeping the cathode current constant eventually results in the deposition of a pure plasma polymer material when the cathode is fully covered or “poisoned” by a plasma polymer layer deposited on the cathode surface. The “poisoning” effect means that the reactive deposition rate from the plasma onto the cathode exceeds the sputtering rate of the coated plasma polymer from the cathode. In this way, the stainless steel cathode can be fully protected from sputtering, resulting in a pure plasma polymer layer deposited onto the substrates.

### Results

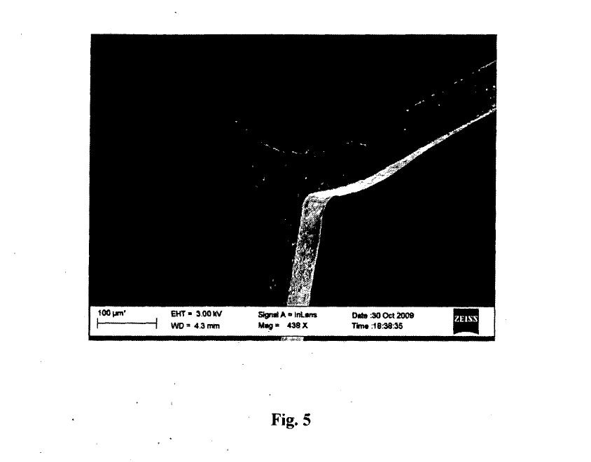

The plasma polymer coating deposited using the above method showed no delamination after crimping and expansion of the stent as show in FIG. 5.

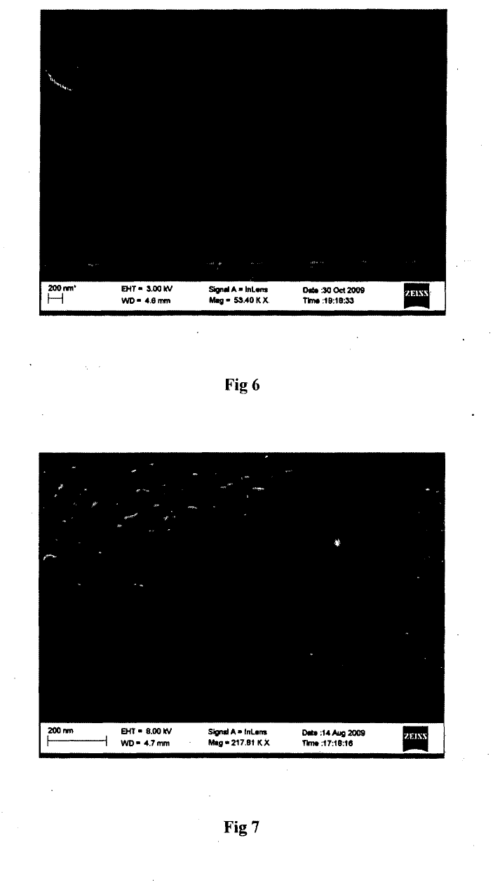

Examination of the layer at higher magnification (FIG. 6) showed the presence of a columnar microstructure. The accommodation of strain through the generation of small gaps between the columns is evident.

At higher magnification of the surface at an area of deformation (FIG. 7) shows more clearly the columnar structure is more clearly visible as are the nanoscale gaps that are formed between columns to accommodate the strain introduced by mechanical deformation of the stent.

More than 50 stents have been coated in accordance with the invention. No adhesion failure has been observed after receiving crimping and expansion as well sequential biological tests both in vitro and in vivo to demonstrate binding of functional biological molecules and resulting biocompatibility.

It is to be understood that the present invention has been described by way of example only and that modifications and/or alterations thereto, which would be apparent to a person skilled in the art based upon the disclosure herein, are also considered to fall within the scope and spirit of the invention, as defined in the appended claims.

1. A deformable implant comprising a metallic substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is a stent that is able to undergo deformation without substantial delamination of the plasma polymer surface.

2. A deformable implant comprising a metallic, polymer and/or composite substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is able to undergo deformation without substantial delamination of the plasma polymer surface.

3. The deformable implant of claim 2 wherein the substrate comprises a composite material.

4. The deformable implant of claim 3 wherein the composite material comprises a metal ceramic composite or a ceramic polymer composite.

5. The deformable implant of claim 2 wherein the substrate comprises metal.

6. The deformable implant of claim 1, wherein the substrate comprises metal alloy.

7. The deformable implant of claim 1, wherein the metal or metal alloy comprises iron, copper, zinc, lead, aluminium, titanium, gold, platinum, silver, cobalt, chromium, vanadium, tantalum, nickel, magnesium, manganese.

8. The deformable implant of claim 6, wherein the metal alloy is cobalt chrome, nickel titanium, titanium vanadium aluminium, stainless steel.

9. The deformable implant of claim 1, wherein the plasma polymer is generated from monomer units selected from one or more of n-hexane, allylamine, acetylene, ethylene, methane, ethanol. butane, propane, pentane, heptane, octane, cyclohexane, cycleoctane, dicyclopentadiene, cyclobutane, tetramethylaniline, methylcyclohexane and ethylcyclohexane, tricyclodecane, propene, allene, pentene, benzene, hexene, octene, cyclohexene, cycloheptene, butadiene, isobutylene, di-para-xylylene, propylene, methylcyclohexane, toluene, p-xylene, m-xylene, o-xylene, styrene, phenol, chlorphenol, chlorbenzene, fluorbenzene, bromphenol, ethylene glycol, diethlyene glycol, dimethyl ether, 2,4,6-trimethyl m-phenylenediamine, furan, thiophene, aniline, pyridine, benzylamine, pyrrole, propionitrole, acrylonitrile, pyrrolidine, butylamine, morpholine, tetrahydrofurane, dimethylformamide, dimethylsulfoxide, glycidyl methacrylate, acrylic acid, ethylene oxide, propylene oxide, ethanol, propanol, methanol, hexanol, acetone, formic acid, acetic acid, tetrafluormethane, fluorethylene, chloroform, tetrachlormethane, trichlormethane, trifluormethane, vinyliden chloride, vinyliden fluoride, hexamethyldisiloxane, triethylsiloxane, dioxane, perfluoro-octane, fluorocyclobutane, octafluorocyclobutane, vinyltriethoxysilane, octafluorotoluene, tetrafluoromethane, hexamethyldisiloxane, heptadecafluoro-1-decene, tetramethyldisilazane, decamethyl-cyclopentasiloxane and perfluoro(methylcyclohexane), 2-chloro-p-xylene.

10. The deformable implant of claim 1, wherein the plasma polymer surface has a thickness of between about 0.3 nm to about 1000 nm.

11. The deformable implant of claim 1, wherein the cross-linked regions are located between about 0.3 nm to about 1000 nm beneath plasma polymer surface.

12. The deformable implant of claim 1, wherein the mixed or graded interface is generated by varying composition of gases supplied to the process chamber or the deposition rate from a metal source so that deposited and/or implanted material changes progressively from more metallic to more polymeric.

13. The deformable implant of claim 1, wherein presence of the plurality of sub-surface cross-linked regions results in delay of plasma polymer surface hydrophobic recovery.

14. The deformable implant of claim 2, which is, or is a component of, a medical device.

15. The medical device of claim 14 selected from a stent, a prosthesis, an artificial joint, a bone or tissue replacement material or scaffold, an artificial organ, a heart valve, a replacement vessel, a suture or a staple.

16. A deformable implantable medical device comprising a columnar structured plasma polymer surface capable of binding functional biological molecules, wherein the device is able to undergo deformation without substantial delamination of the plasma polymer surface.

17. The medical device of claim 15, which is a stent.

18. The deformable implant or medical device of claim 1, wherein columnar structures within the plasma polymer surface have an average diameter of from about 10 nm to about 500 nm.

19. The deformable implant or medical device of claim 1, wherein columnar structures within the plasma polymer surface have an average diameter of from about 20 nm to about 300 nm.

20. The deformable implant or medical device of claim 1, wherein columnar structures within the plasma polymer surface have an average diameter of from about 30 nm to about 200 nm.

21. The deformable implant or medical device of claim 1, wherein the columnar structured plasma polymer surface fully coats the substrate.

22. The deformable implant or medical device of claim 1, wherein the columnar structured plasma polymer surface is located at deformable sections of the substrate.

23. The deformable implant or medical device of claim 1, wherein the plasma polymer surface is directly covalent bound to a functional biological molecule.

24. The deformable implant or medical device of claim 23 wherein the biological molecule comprises one or more amino acids, peptides, proteins, glycoproteins, lipoproteins, nucleotides, oligonucleotides, nucleic acids, lipids and/or carbohydrates.

25. The deformable implant or medical device of claim 23 wherein the biological molecule comprises a drug or drug target.

26. The deformable implant or medical device of claim 23 wherein the biological molecule comprises one or more of antibodies, immunoglobulins, receptors, enzymes, neurotransmitters, cytokines, hormones, complementarity determining proteins, extra-cellular proteins, DNA, RNA and active fragments thereof.

27. The deformable implant or medical device of claim 23 wherein the biological molecule comprises one or more molecules that are integral to or attached to cells or cellular components.

28. The deformable implant or medical device of claim 1, wherein the mixed or graded interface is also columnar structured.

29. A method of producing a deformable implant comprising a metallic, polymer and/or composite substrate, the substrate being bound through a mixed or graded interface to a columnar structured and hydrophilic plasma polymer surface that is activated to enable direct covalent binding to a functional biological molecule, the plasma polymer surface comprising a sub-surface that includes a plurality of cross-linked regions, wherein the implant is able to undergo deformation without substantial delamination of the plasma polymer surface, the method comprising exposing a surface of the substrate to co-deposition under conditions in which substrate material is deposited with carbon containing species while gradually reducing substrate material proportion and increasing carbon containing species proportion, and wherein the co-deposition is conducted under conditions that substantially eliminate energetic ion bombardment.

30. The method of claim 29 further involving incubating the activated columnar structured and hydrophilic plasma polymer surface with a functional biological molecule.

31. The method of claim 29, wherein the mixed or graded interface is also columnar structured.
